(19) United States
(12) Patent Application Publication
(10) Pub. No.: US 2014/0255449 A1
(43) Pub. Date: Sep. 11, 2014

(54) DOSAGE OF DNAK

(71) Applicants: Thierry Legon, Bierbeek (BE); Sabine Pirotton, Brussels (BE); Jean Duchateau, Soignies (BE)

(72) Inventors: Thierry Legon, Bierbeek (BE); Sabine Pirotton, Brussels (BE); Jean Duchateau, Soignies (BE)

(73) Assignee: BioTech Tools S.A., Brussels (BE)

(21) Appl. No.: 14/233,584

(22) PCT Filed: Jul. 19, 2012
(86) PCT No.: PCT/EP2012/064196

§ 371 Date: May 27, 2014

Related U.S. Application Data
(60) Provisional application No. 61/528,857, filed on Aug. 30, 2011.

(30) Foreign Application Priority Data
Jul. 21, 2011 (EP) 11174879.4
Nov. 11, 2011 (EP) 11188763.4

(51) Int. Cl. A61K 39/36; A61K 39/39

(52) U.S. Cl. CPC A61K 39/36; A61K 39/39; A61K 2039/545
USPC 424/275.1

(57) ABSTRACT

A pharmaceutical preparation for subcutaneous injection comprising
between 0.5 ng and 200 μg of HSP70
between 0.5 and 100 μg of fragments of an antigenic structure.

The present invention relates to a pharmaceutical preparation, especially useful for treating allergy, autoimmune disease or graft rejection.

Complexes of peptides together with heat shock proteins are suitable for induction of tolerance.

As an example, U.S. Pat. No. 6,312,711 discloses a pharmaceutical or food composition intended for treating pathologies associated with graft rejection or allergic or autoimmune reaction comprising the administration of a complex of a stress protein and epitopes of an antigenic structure.

Although there have been many developments and improvements in the understanding of tolerance induction, clinical success has not being observed frequently and there are in some cases contradicting results.

### SUMMARY OF THE INVENTION

It is the object of the present invention to provide a pharmaceutical composition having improved and reliable treatment results.

In one embodiment, the present invention provides a pharmaceutical preparation for the treatment of humans comprising
between 0.5 ng and 200 μg of HSP70, preferably 100 μg or less
between 0.5 μg and 100 μg of fragments of an antigenic structure.

In a second embodiment, the present invention provides a method for treating allergy, autoimmune disease or graft rejection comprising administering to a patient cumulated
between 0.5 ng and 200 μg of HSP70
between 0.5 μg and 100 μg of fragments of an antigenic structure.

### DETAILED DESCRIPTION OF THE INVENTION

The present invention is directed to the treatment of allergy by applying small amounts of HSP70 together with fragments of antigenic structures. It is intended to treat humans with the pharmaceutical preparations and the amounts of 0.5 ng to 200 μg HSP and 0.5 μg to 100 μg of antigenic structure are the total amounts applied to the patient at two or more time points, i.e. the pharmaceutical preparation is divided in several doses which are applied to a patient over a certain time period, for example during three to ten administrations.

While a subcutaneous injection is one of the preferred way of administration, the present invention also covers applications via i.e. the nasal route, transdermal patches, oral route, including sublingual application.

Preferably patients are therefore treated with the preparation of the invention in cumulated amounts of 0.5 μg to 100 μg, 1 to 80 μg or 1 to 50 μg or 5 to 25 μg of HSP70 or 0.5 ng to 100 ng or up to 1 μg.

The amount of antigen fragments is between 0.5 μg and 100 μg. Preferred embodiments are between 1 μg to 80 μg or 0.5 μg to 50 μg and 50 μg to 100 μg.

The pharmaceutical preparation could comprise the HSP70/antigen fragments in solution form, this is preferably isotonic. In another form, the pharmaceutical preparation could comprise the HSP70 and the antigen fragments in a freeze-dried powder form.

The pharmaceutical preparation of the present invention may naturally include further ingredients like buffer substances, excipients, adjuvants or the like.

The pharmaceutical preparation of the present invention is especially useful in the treatment of allergy, autoimmune disease or graft rejection.

In a preferred embodiment, the ratio of HSP70: antigen fragments is about 1:1 by weight, but generally a ratio of 2:1 to 1:1000 (w/w) is suitable as well.

Preferred embodiments cover a range between 1:1 and 1:30 or between 1:1 and 1:5. In some embodiments, it is advisable to administer to the patient at each time point the same amount of HSP70, but increasing amounts of antigenic fragments.

In some cases, the HSP70 and the antigen fragment will form complexes. In other embodiments, the HSP70 and the antigenic fragment will not form a complex.

In a preferred embodiment, the antigen fragments are prepared by enzymatic hydrolysis of the antigenic structure, for example using the method described in WO 2008/000783, incorporated by reference.

Preferably, the size of the fragments of an antigenic structure (“antigen fragments”) is between 1000 and 10000 Da.

The antigenic structures are preferably derived from antigenic structures which induce graft rejection, allergic reaction or autoimmune disease. For example for the treatment of grass pollen allergy, the preparation comprises peptides from grass pollen allergens. A preparation for the treatment of peanut allergy would comprise peptide fragments from peanut allergens.

In general, suitable antigenic structures are selected from insulin, thyroglobulin, thyroid peroxidase, type II collagen, gliadin, hordein, secalin, GAD65, proteolipid protein, S-antigen, acetylcholin receptor, haptenized colonic proteins, interphotoreceptor retinoid binding protein, myelin basic protein, myelin oligodendrocyte glycoprotein, peripheral nerve P2, cytoplasmic TSH receptor, intrinsic factor, lens proteins, platelets, nucleoproteins such as histones, heat shock proteins, MHC I, MHC II, MHC-peptides complexes, milk allergens, venom allergens, egg allergens, weed allergens, grass allergens, tree allergens, shrub allergens, flower allergens, grain allergens, fungi allergens, fruit allergens, berry allergens, nut allergens, seed allergens, bean allergens fish allergens, shellfish allergens, meat allergens, spices allergens, insect allergens, mite allergens, animal allergens, animal dander allergens, allergens of Hevea brasiliensis, coagulation factors and blood group antigens.

HSP70 covers eukaryotic and prokaryotic heat shock proteins of about 70 kDa. For review on heat shock proteins see Van Eden et al., Nature Reviews, Immunology 5 (2005), 318-330.

A preferred method of preparing DnaK is described in WO 2008/043832, incorporated by reference.

A preferred HSP70 of the present invention is a prokaryotic HSP70. Saprophytic prokaryotic HSP70 are especially preferred. A very preferred HSP70 is E. coli DnaK. HSP70 is a compound which is able to bind ATP and ADP and has an ATPase activity. This activity might be influenced by the amount of phosphate in the pharmaceutical preparation.

In some embodiments the concentration of phosphate in the buffer should be preferably not more than 50 mmol/l, preferably less than 20 mmol/l or less than 3 mmol/1 or less than 2 mmol/l.

In preferred embodiments, the total amount of the pharmaceutical preparation is administered in at least two time points, but the use in 3 to 5, or 3 to 10 administrations is generally preferred.

In some embodiments of the invention, the pharmaceutical preparation could be one vial which comprises the complete amount of HSP70/antigen fragments, for example 50 μg of antigen peptides and 50 μg of HSP70. For treatment suitable amounts are taken from the vial. For example, the vial could comprise a total amount of 100 μg in 1.5 ml solution and administrations could be conducted with 100 μl, 200 μl, 400 μl, 800 μl with one week intervals. Typically, for subcutaneous administration the amount should be not more than 1000 μl.

In other embodiments, the pharmaceutical preparation could comprise separate vials comprising the required amount of HSP70/antigen fragments for one administration, for example four vials comprising 20 μg HSP70/antigen fragments.

In other embodiments, the pharmaceutical preparation could comprise separate medical devices comprising the necessary amounts, for example four syringes comprising 100, 200, 400 and 800 μl according to the administration scheme.

Instead of the syringes, preparations for oral administration like tablets or for nasal administration could be used. For a nasal administration one could use increased number of puffs, for example 1, 2, 4 puffs wherein each puff comprises for example 5 μg HSP70 and 5 μg of antigen fragments.

In a further embodiment, it would be possible to use patches for the transdermal application during treatment. Either the number or the size of the patches could be increased to increase at different time points the amount of substance applied.

In another embodiment, the syringes could comprise the same amount of solution, but in different concentrations, for example the solution in the syringe could always be 200 μl, but the amount of HSP70/antigen fragments could be 5/5 μg, 10/10 μg, 20/20 μg. As mentioned before, it could also be possible not to change the amount of HSP and to apply for example 5/5 μg, 5/10 μg, 5/20 μg (HSP70/antigenic fragment) and the like.

Subject matter of the present invention is also the method of treating allergy, autoimmune disease or graft rejection comprising the step of administering the pharmaceutical preparation of the present administration, preferably in at least 2, preferably 3 to 10 or 3 to 5 administrations.

In preferred embodiments, the time between two administrations is about 5 to 10 days.

Injection and preferably subcutaneous injection is a preferred way of administration.

The inventors have conducted a phase one double-blind placebo-controlled prospective randomized comparative study. As an example, a standard of grass pollen allergen fragments was administered alone or with an equal weight of DnaK by subcutaneous injection.

### BRIEF DESCRIPTION OF THE FIGURES

FIGS. 1a and 1b show the number of well days for peptides alone and peptides+DnaK

FIG. 2 shows the changes for IgG4.

FIG. 3 shows the changes for IgE.

FIG. 4 shows the number of well days for different groups.

### EXAMPLES

The Study

27 subjects were divided in three groups:

A) Placebo

B) Grass pollen allergen fragments

C) Grass pollen allergen fragments+DnaK.

The intention was to treat the groups with 5 subcutaneous injections at 1 week's interval according to the following table:

| | | | C) Allergen |
|---|---|---|---|
| | A) Placebo | B) Allergen fragments | fragments/DnaK |
| Inj. 1 (D1) | placebo | 5 μg | 5 μg + 5 μg |
| Inj. 2 (D8) | placebo | 10 μg | 10 μg + 10 μg |
| Inj. 3 (D15) | placebo | 20 μg | 20 μg + 20 μg |
| Inj. 4 (D22) | placebo | 50 μg | 50 μg + 50 μg |
| Inj. 5 (D29) | placebo | 50 μg | 50 μg + 50 μg |

Depending on the local reactions after the injection of the principle investigators had the possibility not to increase the dosage in the next week.

Three subjects in each groups B) and C) received therefore less than 105 μg during treatment.

Results

Clinical Efficiency

Number of Well Days

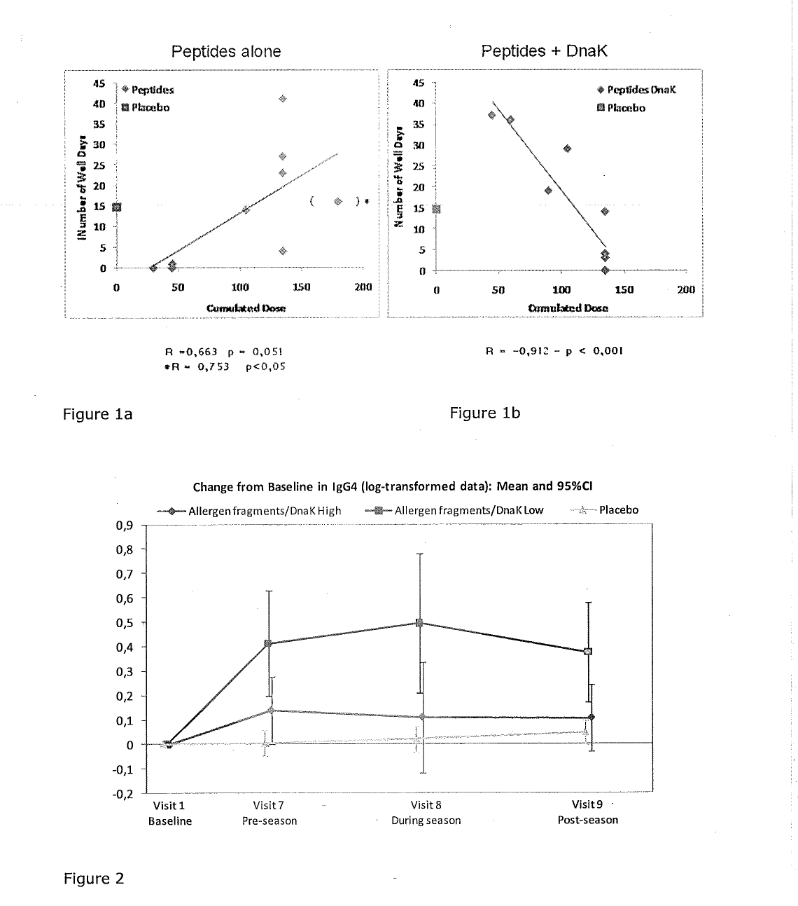

The relation between cumulated dose and well days (day with a rhinoconjonctivitis Score <2 and no rescue medication intake) is shown in FIGS. 1a and 1b.

FIG. 1a is shows the number of well days in relation to the cumulated doses in group B). As can be derived from the results, there is a relation between number of well days and the amount of cumulated dose.

Interestingly, as shown in FIG. 1b) for group C) the number of well days decreased with the cumulated dose, i.e. an improvement of the number of well days was observed for a smaller amount of grass pollen allergen fragments and DnaK and this result was significantly better than the results of Placebo group A).

Induction of DnaK Specific IgG Antibodies

In contrast to the group treated with high amounts of cumulated dose of allergen fragments/DnaK, a treatment with low amounts of allergen fragments/DnaK did not induce DnaK specific IgG production.

Induction of Allergen Specific IgG4 and IgE

A high level of grass pollen specific IgG4 before a pollen season—whether naturally acquired or stimulated by immunotherapy—is usually considered as a surrogate marker for the alleviation of seasonal allergic rhinitis (SAR) symptoms during the subsequent season.

Grass pollen-specific IgE and IgG4 are induced in parallel at the beginning of the treatment.

Only a prolonged immunization has been previously shown to result in an increase in grass pollen-specific IgG4 and in a decrease fall in specific IgE.

Interestingly, the group treated with low cumulated doses of grass pollen fragments and DnaK showed a clear increase in IgG4 anti-pollen antibodies in contrast to the group treated with higher cumulated doses; see FIG. 2.

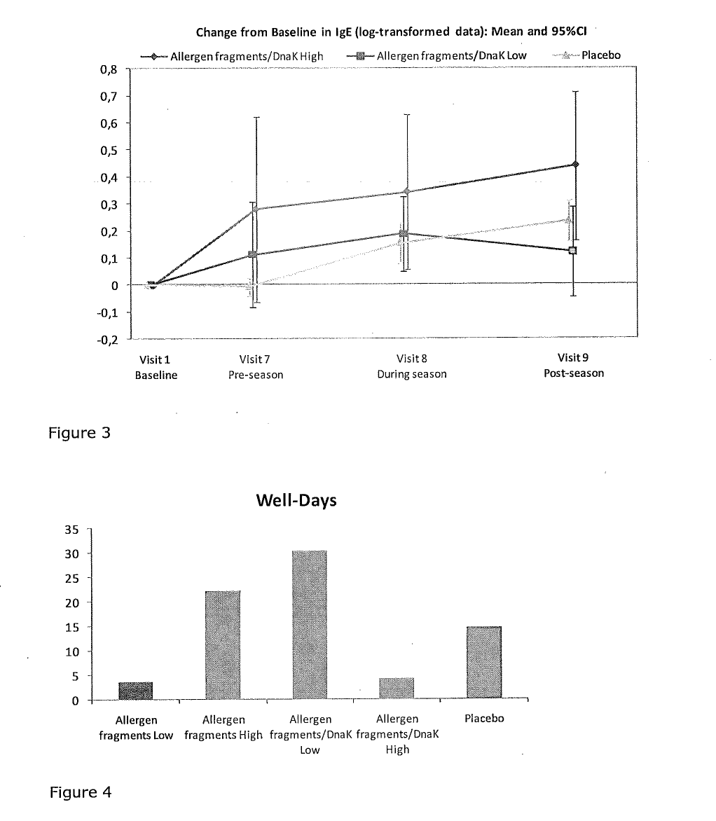

In contrast thereto, the anti-pollen IgE increased stronger in the group with high amounts of grass pollen allergen fragments and DnaK and was lower for the low cumulated dose treated group; see FIG. 3.

Scores

Additionally, four further parameters, namely RhinoConjunctivitis Score (RCS), Rescue Medication Score (RMS), Total Symptom Score (TSS) and Average Combined Score (ACS) were recorded in addition to number of well days.

The results for group C) can be found in table 2.

| Treatment | RCS | RMS | TSS | ACS | Well Days |
|---|---|---|---|---|---|
| Low dose DnaK | 1.64 | 0.20 | 2.16 | 0.24 | 30.25 |
| High dose DnaK | 6.29 | 0.70 | 7.47 | 0.88 | 4.20 |
| T-test Low vs High | 0.06 | 0.04 | 0.14 | 0.04 | 0.003 |
| Placebo | 3.49 | 0.37 | 3.90 | 0.49 | 14.67 |
| T-test Placebo vs Low dose | 0.16 | 0.37 | 0.34 | 0.23 | 0.02 |
| T-test Placebo vs High dose | 0.29 | 0.11 | 0.26 | 0.15 | 0.03 |

The difference between low cumulated dose treatment of grass pollen fragments/DnaK and high doses treatment with grass pollen fragments/DnaK, as well as the differences between both groups and the Placebo group are statistically significant.

Table 3 shows the results for treatment group B.

| Treatment | RCS | RMS | TSS | ACS | Well Days |
|---|---|---|---|---|---|
| Low dose pept | 6.87 | 0.60 | 8.47 | 0.90 | 0.33 |
| High dose pept | 2.47 | 0.22 | 2.63 | 0.30 | 20.83 |
| T-test Low vs High | 0.04 | 0.26 | 0.04 | 0.002 | 0.01 |
| Placebo | 3.49 | 0.37 | 3.90 | 0.49 | 14.67 |
| T-test Placebo vs Low dose | 0.09 | 0.46 | 0.08 | 0.005 | 0.002 |
| T-test Placebo vs High dose | 0.38 | 0.30 | 0.30 | 0.23 | 0.34 |

In summary, looking at the number of well days the low dose of the grass pollen fragments/DnaK gave a better result than the high dose of grass pollen fragments alone; see FIG. 4.

In summary, the patients had the greatest benefit if they received less than 100 μg and preferably less than 50 μg DnaK/and less than 100 μg and preferably less than 50 μg fragments of an antigenic structure.

Pharmaceutical Preparation

Heat shock protein was prepared by a method described in WO 2008/043832 and grass pollen antigen was prepared according to the method of WO 2008/000783.

A solution comprising 100 μg per ml of both products was prepared comprising 12 mM NaCl, 5 mM sodium phosphate, 4.2% (w/v) manitol, 1% (w/v) trehalose. The solution was distributed in vials comprising increasing amounts of HSP and antigen (in the amount of 25, 50, 100, 200 and 400 μl). The vials were frozen and lyophilized. Suitable doses could be prepared by reconstituting the lyophilized pharmaceutical preparation. They were used for 5 administrations in one week intervals. The amount was increased when the previous injection was well tolerated.

1. A pharmaceutical preparation for subcutaneous injection comprising
between 0.5 ng and 200 μg of HSP70
between 0.5 and 100 μg of fragments of an antigenic structure.

2. The pharmaceutical preparation of claim 1, comprising 80 μg or less, 50 μg or less, or 25 μg or less of HSP70.

3. The pharmaceutical preparation of claim 1, in the form of a solution or a freeze-dried powder.

4. The pharmaceutical preparation of claim 1, for use in the treatment of allergy, autoimmune disease or graft rejection.

5. The pharmaceutical preparation of claim 1, wherein the ratio of HSP70: fragments of an antigenic structure is between 2:1 and 1:1000 (w/w), preferably between 1:1 and 1:30 (w/w) or between 1:1 and 1:5 (w/w).

6. The pharmaceutical preparation of claim 1, wherein the fragments of an antigenic structure are prepared by enzymatic hydrolysis of the antigenic structure.

7. The pharmaceutical preparation of claim 1, wherein the antigenic structures are selected from antigenic structures which induce graft rejection, allergic reaction or autoimmune disease, preferably wherein the antigenic structures are selected from insulin, thyroglobulin, thyroid peroxidase, type II collagen, gliadin, hordein, secalin, GAD65, proteolipid protein, S-antigen, acetylcholin receptor, haptenized colonic proteins, interphotoreceptor retinoid binding protein, myelin basic protein, myelin oligodendrocyte glycoprotein, peripheral nerve P2, cytoplasmic TSH receptor, intrinsic factor, lens proteins, platelets, nucleoproteins such as histones, heat shock proteins, MHC I, MHC II, MHC-peptides complexes, milk allergens, venom allergens, egg allergens, weed allergens, grass allergens, tree allergens, shrub allergens, flower allergens, grain allergens, fungi allergens, fruit allergens, berry allergens, nut allergens, seed allergens, bean allergens fish allergens, shellfish allergens, meat allergens, spices allergens, insect allergens, mite allergens, animal allergens, animal dander allergens, allergens of Hevea brasiliensis, coagulation factors and blood group antigens.

8. The pharmaceutical preparation of claim 1 wherein the pharmaceutical preparation is divided in 3 to 10 doses.

9. The pharmaceutical preparation of claim 8 wherein the doses have a different amount of fragments of antigenic structure.

10. The pharmaceutical preparation of claim 1, wherein the preparation is for use in a treatment comprising at least two injections in a patient at different time points, preferably wherein the preparation is for use in a treatment comprising of 3 to 5 injections with increasing amounts of the preparation.

11. The pharmaceutical preparation of claim 1 wherein the HSP70 is a prokaryotic HSP70, preferably a saprophytic prokaryotic HSP70 and most preferably E. Coli DnaK.

12. A method of treating allergy, autoimmune disease or graft rejection comprising administering to a patient by subcutaneous injection a cumulated dose of
between 0.5 ng and 200 μg of HSP70
between 0.5 and 100 μg of fragments of an antigenic structure.

13. The method of claim 12, wherein the preparation is divided in at least two injections.

14. The method of claim 12, wherein the preparation is divided in three to five injections, preferably wherein the time interval between two injections is 5 to 10 days.

15. The method of claim 12 wherein the HSP70 is a prokaryotic HSP70, preferably a saprophytic prokaryotic HSP70 and most preferably E. Coli DnaK.
